(19) United States
(12) Patent Application Publication
(10) Pub. No.: US 2015/0051473 A1
(43) Pub. Date: Feb. 19, 2015

(54) SYSTEMS AND METHODS FOR PHOTOACOUSTIC SPECTROSCOPY

(71) Applicant: Covidien LP, Mansfield, MA (US)

(72) Inventors: Qiaojian Huang, Broomfield, CO (US); Charles Haisley, Boulder, CO (US); Sarah Hayman, Boulder, CO (US); Youzhi Li, Longmont, CO (US); Friso Schlottau, Lyons, CO (US)

(73) Assignee: Covidien LP, Mansfield, MA (US)

(21) Appl. No.: 13/968,129

(22) Filed: Aug. 15, 2013

(51) Int. Cl. A61B 5/00; A61B 5/1455; A61B 5/024

(52) U.S. Cl. CPC A61B 5/0095; A61B 5/02444; A61B 5/14552
USPC 600/407

(57) ABSTRACT

A photoacoustic sensor system includes a photoacoustic sensor assembly having a light emitting component configured to emit one or more wavelengths of light into a region of a patient's tissue and an acoustic detector configured to detect acoustic energy generated within the region of the patient's tissue in response to the emitted light. The photoacoustic sensor assembly is configured to generate a signal that enables a monitor to determine a potion of the sensor assembly relative to the patient's tissue.

### BACKGROUND

The present disclosure relates generally to medical devices and, more particularly, to the use of photoacoustic spectroscopy in patient monitoring.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological characteristics of their patients. Accordingly, a wide variety of devices have been developed for monitoring patient characteristics. Such devices provide doctors and other healthcare personnel with the information they need to provide healthcare for their patients. As a result, such monitoring devices have become an indispensable part of modern medicine. For example, in certain medical contexts, it may be desirable to ascertain various localized physiological parameters, such as parameters related to individual blood vessels or other discrete components of the vascular system. Examples of such parameters may include oxygen saturation, hemoglobin concentration, perfusion, and so forth, for an individual blood vessel.

In one approach, measurement of such localized parameters is achieved via photoacoustic (PA) spectroscopy. PA spectroscopy utilizes light directed into a patient's tissue to generate acoustic waves that may be detected and resolved to determine localized physiological information of interest. In particular, the light energy directed into the tissue may be provided at particular wavelengths that correspond to the absorption profile of one or more blood or tissue constituents of interest. In some systems, the light is emitted as pulses (i.e., pulsed PA spectroscopy), though in other systems the light may be emitted in a continuous manner (i.e., continuous PA spectroscopy). The light absorbed by the constituent of interest results in a proportionate increase in the kinetic energy of the constituent (i.e., the constituent is heated), which results in the generation of acoustic waves. The acoustic waves may be detected and used to determine the amount of light absorption, and thus the quantity of the constituent of interest, in the illuminated region. For example, the detected acoustic energy may be proportional to the optical absorption coefficient of the blood or tissue constituent and the fluence of light at the wavelength of interest at the localized region being interrogated (e.g., a specific blood vessel).

### BRIEF DESCRIPTION OF THE DRAWINGS

Advantages of the disclosed techniques may become apparent upon reading the following detailed description and upon reference to the drawings in which:

FIG. 1 is a block diagram of a patient monitor and a photoacoustic sensor assembly in accordance with an embodiment;

FIG. 2 is a schematic illustrating a photoacoustic sensor assembly having an angled light delivery system, in accordance with an embodiment;

FIG. 3 is a schematic illustrating a photoacoustic sensor assembly having an orthogonal light delivery system, in accordance with an embodiment;

FIG. 4 is a schematic illustrating an ear clip style photoacoustic sensor assembly, in accordance with an embodiment;

FIG. 5 is a schematic of a photoacoustic sensor assembly having an optical detector, in accordance with an embodiment;

FIG. 6 is a plot of an optical signal obtained as a sensor assembly transitions from a position on a patient's skin to a position off of the patient's skin, in accordance with an embodiment;

FIG. 7 is a flow diagram of a method for determining an appropriate response based on whether a photoacoustic sensor assembly is applied to a patient's tissue, in accordance with an embodiment; and

FIG. 8 is flow diagram of a method for determining an appropriate response based on whether a photoacoustic sensor assembly is applied to a patient's skin and a current mode of operation, in accordance with an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Presently disclosed embodiments of photoacoustic (PA) sensors, systems, and methods are provided for the measurement of various localized physiological parameters, such as parameters related to individual blood vessels or other discrete components of the vascular system. Examples of such parameters may include but are not limited to oxygen saturation, hemoglobin concentration, perfusion, cardiac output, and so forth, for an individual blood vessel. In certain embodiments, the disclosed PA sensors may be utilized as part of a PA spectroscopy system in which light is directed into a patient's tissue to generate acoustic waves that may be detected and resolved to determine the localized physiological information of interest. In these embodiments, the light energy directed into the tissue is provided at particular wavelengths that correspond to the absorption profile of one or more blood or tissue constituents of interest. In some embodiments, the PA spectroscopy system may additionally or alternatively be used to non-invasively measure indicator dilution, which may relate to cardiac output and other hemodynamic parameters. For example, for patients with an indicator solution injected into a vein, PA monitoring techniques may be used to measure dilution of the indicator in a downstream artery after mixing in the blood. Disclosed embodiments may be utilized in PA spectroscopy systems in which the light is emitted as pulses (i.e., pulsed PA spectroscopy), as well as in systems in which the light is emitted in a continuous manner (i.e., continuous PA spectroscopy). Additionally, the disclosed embodiments may be used and/or combined with the PA sensors and monitoring systems disclosed in U.S. patent application Ser. No. 13/842,466 filed on Mar. 15, 2013, the specification of which is incorporated by reference herein in its entirety herein for all purposes.

Proper PA sensor placement is associated with improved measurements. The use of high-intensity light sources, such as lasers, in PA spectroscopy may also involve specialized training and equipment, which may be complex and costly. Thus, certain features of the disclosed embodiments may mitigate the effects of high-intensity light sources in the PA sensor. For example, in certain embodiments, the PA sensor includes a light emitter, such as a laser diode, oriented at an angle such that the emitted light is totally internally reflected when the sensor is exposed to air, to reduce the intensity of the emitted light when the sensor is away from a patient's skin. Accordingly, when the sensor is removed from the tissue, little to no light is emitted. Additionally, in certain embodiments, the associated system may monitor an acoustic signal to detect unexpected changes or diminishment of the acoustic signal. Unexpected change or diminishment of the acoustic signal may indicate that the sensor is improperly placed on or removed from the patient's tissue. In some embodiments, the PA sensor may include an optical detector for detecting light emitted by the PA light source. Characteristics of the detected optical signal may indicate whether the sensor is appropriately placed on the subject's skin. For example, the detected optical signal may increase when the sensor is removed from the skin due to reflection of the light off the skin toward the optical detector. Additionally or alternatively, in certain embodiments, the PA sensor includes one or more contact sensing elements, which may generate signals indicative of whether the PA sensor is applied to the patient's skin, for example.

As described in more detail below, the foregoing features may facilitate the reduction in the emission of high-intensity light when the PA sensor is not applied to the patient's skin. In some embodiments, the PA spectroscopy system may alter characteristics (e.g., power, repetition rate, pulse width) of the emitted light based at least in part on whether the PA is applied to the patient's skin, as discussed in more detail below.

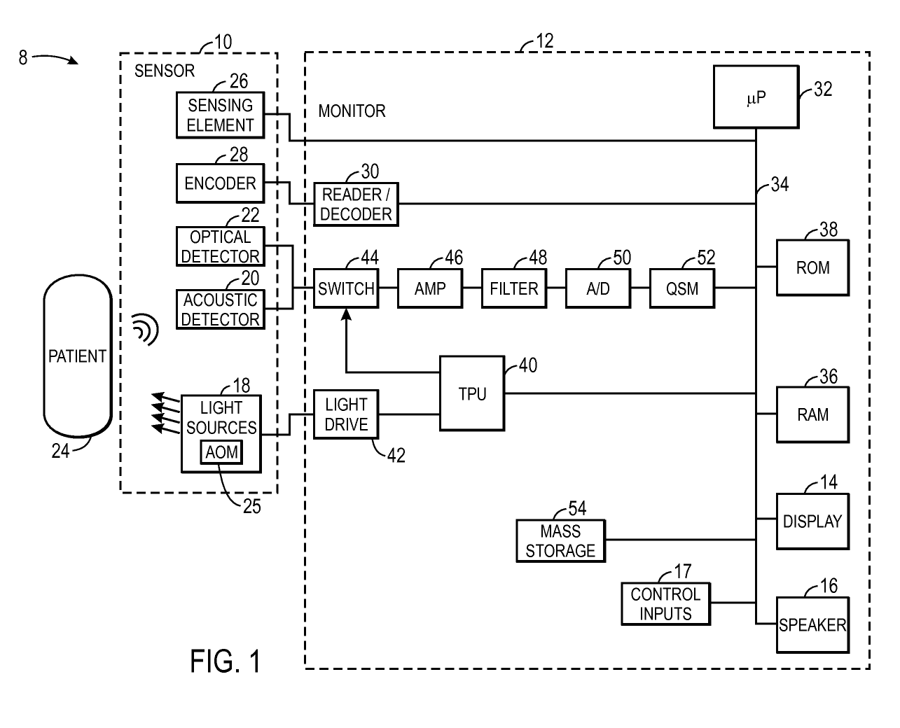

With this understanding, FIG. 1 depicts a block diagram of a photoacoustic spectroscopy system 8 in accordance with embodiments of the present disclosure. The system 8 includes a photoacoustic spectroscopy sensor 10 and a monitor 12. During operation, the sensor 10 emits light at certain wavelengths into a patient's tissue and detects acoustic shock waves (e.g., ultrasound waves) generated in response to the emitted light. The monitor 12 is capable of calculating physiological characteristics based on signals received from the sensor 10 that correspond to the detected acoustic shock waves. The monitor 12 may include a display 14 and/or a speaker 16, which may be used to convey information about the calculated physiological characteristics to a user. The monitor 12 may be configured to receive user inputs via control inputs 17. The sensor 10 may be communicatively coupled to the monitor 12 via a cable or, in some embodiments, via a wireless communication link.

In one embodiment, the sensor 10 may include a light source 18 and an acoustic detector 20, such as an ultrasound transducer. The light source 18 may emit light as pulses or in a continuous manner. Further, in certain embodiments, the light source 18 may be associated with one or more optical fibers for conveying light from one or more light generating components to the tissue site. In some embodiments, the sensor 10 may also include an optical detector 22 that may be a photodetector, such as a silicon photodiode package, selected to receive light in the range emitted from the light source 18. In the present context, the optical detector 22 may be referred to as a detector, a photodetector, a detector device, a detector assembly or a detector component. Further, the optical detector 22 and light source 18 may be referred to as optical components or devices. In some embodiments, the optical detector 22 is configured to receive light emitted by the light source 18, although in other embodiments, the sensor 10 may include an additional light source for use with the optical detector 22.

The sensor 10 may include the light source 18 and the acoustic detector 20 that may be of any suitable type. For example, in one embodiment, the light source 18 may include one, two, or more light emitting components (such as light emitting diodes or LEDs) adapted to transmit light at one or more specified wavelengths. In certain embodiments, the light source 18 may include a laser diode or a vertical cavity surface emitting laser (VCSEL). The laser diode may be a tunable laser, such that a single diode may be tuned to various wavelengths corresponding to a number of different absorbers of interest in the tissue and blood. That is, the light may be any suitable wavelength or wavelengths (such as a wavelength between about 500 nm to about 1000 nm or between about 600 nm to about 900 nm) that is absorbed by a constituent of interest in the blood or tissue. For example, wavelengths between about 500 nm to about 600 nm, corresponding with green visible light, may be absorbed by deoxyhemoglobin and oxyhemoglobin. In other embodiments, red wavelengths (e.g., about 600 nm to about 700 nm) and infrared or near infrared wavelengths (e.g., about 800 nm to about 1000 nm) may be used. In one embodiment, the selected wavelengths of light may penetrate into the tissue of a patient 24 up to approximately 1 mm to approximately 3 cm. In certain embodiments, the selected wavelengths may penetrate through bone (e.g., the rib cage) of the patient 24. In disclosed embodiments that include the light source 18, it should be understood that the light source 18 may be coupled to an optical fiber.

To increase the precision of the measurements, the emitted light may be focused on an internal region of interest by modulating the intensity and/or phase of the illuminating light. Accordingly, an acousto-optic modulator (AOM) 25 may modulate the intensity of the emitted light, for example, by using linear frequency modulation (LFM) techniques. The emitted light may be intensity modulated by the AOM 25 or by changes in the driving current of the LED emitting the light. The intensity modulation may result in any suitable frequency, such as from 1 MHz to 10 MHz or more. Accordingly, in one embodiment, the light source 18 may emit LFM chirps at a frequency sweep range approximately from 1 MHz to 5 MHz. In another embodiment, the frequency sweep range may be of approximately 0.5 MHz to 10 MHz. The frequency of the emitted light may be increasing with time during the duration of the chirp. In certain embodiments, the chirp may last approximately 0.1 second or less and have an associated energy of a 10 mJ or less, such as between 1 μJ to 2 mJ, 1-5 mJ, 1-10 mJ. In such an embodiment, the limited duration of the light may prevent heating of the tissue while still emitting light of sufficient energy into the region of interest to generate the desired acoustic waves when absorbed by the constituent of interest.

In disclosed embodiments, the acoustic waves may be detected by the acoustic detector 20, which may include an ultrasound transducer or transducer array. In one example, the acoustic detector 20 may be one or more ultrasound transducers, such as a focused ultrasound transducer, suitable for detecting ultrasound waves emanating from the tissue in response to the emitted light and for generating a respective optical or electrical signal in response to the ultrasound waves. For example, the acoustic detector 20 may be suitable for measuring the frequency and/or amplitude of the acoustic waves, the shape of the acoustic waves, and/or the time delay associated with the acoustic waves with respect to the light emission that generated the respective waves. In one embodiment an acoustic detector 20 may be an ultrasound transducer employing piezoelectric or capacitive elements to generate an electrical signal in response to acoustic energy emanating from the tissue of the patient 24, i.e., the transducer converts the acoustic energy into an electrical signal. The acoustic detector 20 may be made, for example, of piezoelectric materials such as lead zirconate titanate (PZT), polyvinylidene fluoride (PVDF), and so forth.

In some embodiments, the system 8 may also include any number or combination of additional medical sensors for providing information related to patient parameters that may be used in conjunction with the PA spectroscopy sensor 10. For example, suitable additional medical sensors may include sensors for determining blood pressure, blood constituents, respiration rate, respiration effort, heart rate, patient temperature, cardiac output, and so forth. Such information may be used, for example, to determine if the patient is in shock or has an infection. Additionally, the system 8 may also include one or more contact sensing elements 26 (e.g., sensing elements) configured to generate signals or to provide information related to whether the sensor 10 is applied to the patient's skin. Such sensing elements 26 may be included within or may be coupled to the sensor 10, or may otherwise be incorporated into or be in communication with the system 8. As described in detail below, the sensing elements 26 may include an impedance sensor or a temperature sensor, for example.

The sensor 10 may include a memory or other data encoding component, depicted in FIG. 1 as an encoder 28. For example, the encoder 28 may be a solid state memory, a resistor, or combination of resistors and/or memory components that may be read or decoded by the monitor 12, such as via reader/decoder 30, to provide the monitor 12 with information about the attached sensor 10. For example, the encoder 28 may encode information about the sensor 10 or its components (such as information about the light source 18 and/or the acoustic detector 20). Such encoded information may include information about the configuration or location of photoacoustic sensor 10, information about the type of lights source(s) 18 present on the sensor 10, information about the wavelengths, light wave frequencies, chirp durations, and/or light wave energies which the light source(s) 18 are capable of emitting and the properties and/or detection range of the optical detector 22, information about the nature of the acoustic detector 20, and so forth. In certain embodiments, the information also includes a reference LFM chirp that was used to generate the actual LFM emitted light. This information may allow the monitor 12 to select appropriate algorithms and/or calibration coefficients for calculating the patient's physiological characteristics, such as the amount or concentration of a constituent of interest in a localized region, such as a blood vessel.

In one implementation, signals from the acoustic detector 20 (and decoded data from the encoder 28, if present) and the optical detector 22 may be transmitted to the monitor 12. The monitor 12 may include data processing circuitry (such as one or more processors 32, application specific integrated circuits (ASICS), or so forth) coupled to an internal bus 34. Also connected to the bus 34 may be a RAM memory 36, a ROM memory 38, a speaker 16 and/or a display 14. In one embodiment, a time processing unit (TPU) 40 may provide timing control signals to light drive circuitry 42, which controls operation of the light source 18, such as to control when, for how long, and/or how frequently the light source 18 is activated, and if multiple light sources are used, the multiplexed timing for the different light sources.

The TPU 40 may also control or contribute to operation of the acoustic detector 20 and/or the optical detector 22 such that timing information for data acquired using the acoustic detector 20 and/or the optical detector 22 may be obtained. Such timing information may be used in interpreting the acoustic wave data and/or in generating physiological information of interest from such acoustic data. For example, the timing of the acoustic data acquired using the acoustic detector 20 may be associated with the light emission profile of the light source 18 during data acquisition. Likewise, in one embodiment, data acquisition by the acoustic detector 20 may be gated, such as via a switching circuit 44, to account for differing aspects of light emission. For example, operation of the switching circuit 44 may allow for separate or discrete acquisition of data that corresponds to different respective wavelengths of light emitted at different times. Similarly, the data acquired from the optical detector 22 may be gated via the switched circuit 44.

The received signal from the acoustic detector 20 and/or the optical detector 22 may be amplified (such as via amplifier 46), may be filtered (such as via filter 48), and/or may be digitized if initially analog (such as via an analog-to-digital converter 50). The digital data may be provided directly to the processor 32, may be stored in the RAM 36, and/or may be stored in a queued serial module (QSM) 52 prior to being downloaded to RAM 36 as QSM 52 fills up. In one embodiment, there may be separate, parallel paths for separate amplifiers, filters, and/or A/D converters provided for different respective light wavelengths or spectra used to generate the acoustic data. Further, while the disclosed block diagram shows the signal from the optical detector 22 and the acoustic detector 20 being supplied to the same path (e.g., a path that may include a switch 44, amplifier 46, filter 48, A/D converter 50, and/or a QSM 52), it should be understood that these signals may be received and processed on separate paths or separate channels.

The data processing circuitry, such as processor 32, may derive one or more physiological characteristics based on data generated by the sensor 10. For example, based at least in part upon data received from the acoustic detector 20, the processor 32 or other suitable circuitry may calculate the amount or concentration of a constituent of interest in a localized region of tissue or blood using various algorithms. In one embodiment, the processor 32 may calculate one or more of cardiac output, total blood volume, extravascular lung water, intrathoracic blood volume, systemic and pulmonary blood flow, and/or macro and microvascular blood flow from signals obtained from a signal sensor 10. In certain embodiments, these algorithms may use coefficients, which may be empirically determined, that relate the detected acoustic waves generated in response to emitted light waves at a particular wavelength or wavelengths to a given concentration or quantity of a constituent of interest within a localized region.

In one embodiment, the processor 32 may access and execute coded instructions, such as for implementing the algorithms discussed herein, from one or more storage components of the monitor 12, such as the RAM 36, the ROM 38, and/or a mass storage 54. Additionally, the RAM 36, ROM 38, and/or the mass storage 54 may serve as data repositories for information such as templates for LFM reference chirps, coefficient curves, and so forth. For example, code encoding executable algorithms may be stored in the ROM 38 or mass storage device 54 (such as a magnetic or solid state hard drive or memory or an optical disk or memory) and accessed and operated according to processor 32 instructions using stored data. Such algorithms, when executed and provided with data from the sensor 10, may calculate one or more physiological characteristics as discussed herein (such as the concentration or amount of a constituent of interest). Once calculated, the physiological characteristics may be displayed on the display 14 for a caregiver to monitor or review.

With the foregoing system discussion in mind, light emitted by the light source 18 of the sensor 10 may be directed into a patient's tissue to generate acoustic signals in proportion to the amount of an absorber (e.g., a constituent of interest, such as a saline indicator) in a targeted localized region. However, as noted above, one problem that may arise in photoacoustic spectroscopy is that the light source 18 may emit a high-intensity light, and it may be desirable to control the emission of the high-intensity light for improved measurements, for example. Accordingly, certain embodiments of the present disclosure relate to PA spectroscopy systems 8 configured so that the high-intensity light emitted by the light source 18 is totally internally reflected within the sensor 10 if the sensor 10 is surrounded by air (e.g., not on the patient's skin), or so that the light source 18 emits light only if the sensor 10 is positioned on the patient's skin. Additionally, certain embodiments of the present disclosure relate to PA spectroscopy systems 8 having various configurations and/or features that enable the system 8 to determine whether the sensor 10 is positioned on the patient's skin. The system 8 may, in turn, be configured to adjust or control emission of light from the light source 18 based at least in part on whether the sensor is positioned on the patient's skin. Examples of suitable configurations of the sensor 10 are discussed in more detail below.

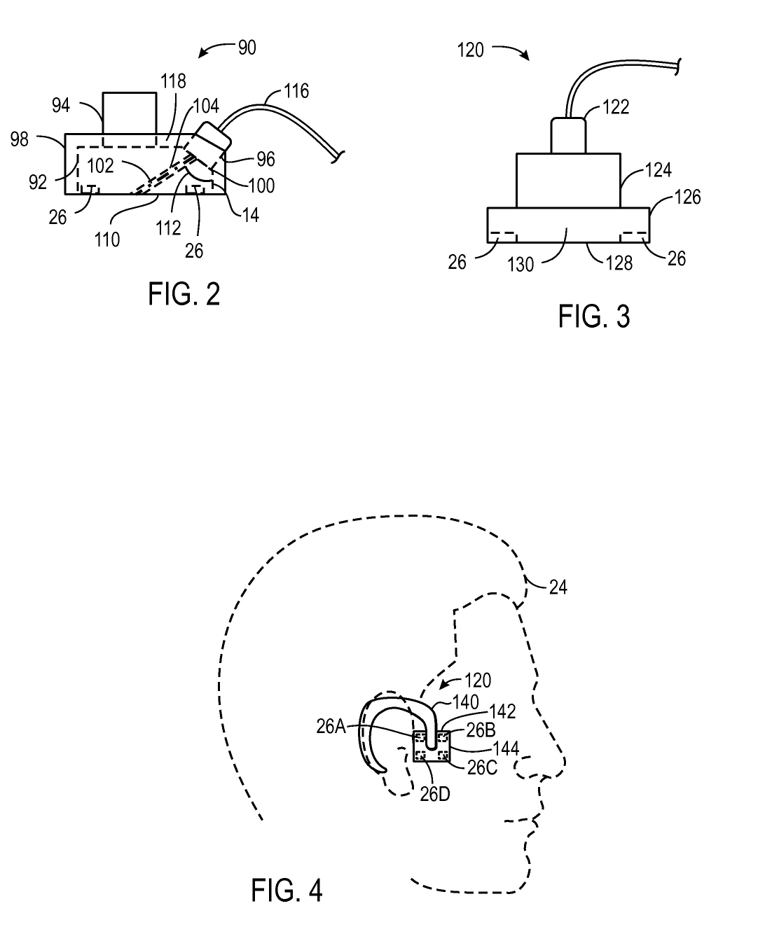

FIG. 2 illustrates an embodiment of a PA sensor assembly 90 including an optically transparent and ultrasound coupling spacer 92, an acoustic detector 94, a laser diode assembly 96 (e.g., an angled laser diode assembly), and a housing 98. The angled laser diode assembly 96 is configured to emit light into the spacer 92 for patient monitoring. In certain embodiments, the spacer 92 is a Rexolite prism. Rexolite is utilized as the spacer 92 in some embodiments because of its low ultrasound attenuation, high light transmission, and its ability to be machined to the prism shape, which facilitates tuning of the direction of ultrasound propagation during operation of the sensor assembly 90. However, in other embodiments, any desired spacer 92 having any desired features may be employed, not limited to Rexolite, depending on implementation-specific considerations. For instance, in some embodiments, the spacer 92 may be any material having a low ultrasound impedance (i.e., an ultrasound impedance approximately equal or close to the ultrasound impedance of the tissue of the patient) and high light transmission. For example, in one embodiment, the ultrasound impedance of the spacer 92 may be approximately 1.5-1.6 MRayls. Additionally, the sensor assembly 90 may include any suitable acoustic detector 94.

As shown in FIG. 2, in certain embodiments, the angled laser diode assembly 96 may be positioned so that light is delivered at a non-orthogonal angle relative to the patient's tissue when the sensor 10 is in operation. The spacer 92 includes an angled face or portion 100 that accommodates the angled laser diode assembly 96. In some embodiments, the angled laser diode assembly 96 may transmit light into an angled optical fiber 102 disposed within an angled optical channel 104, although in other embodiments, the angled laser diode assembly 96 emits light into the spacer 92 without the use of the angled optical fiber 102 or the angled optical channel 104. During operation, the sensor assembly 90 is positioned with respect to the patient such that a bottom surface 110 of the spacer 92 is in contact with the surface of the patient's tissue. Once the light is emitted and transmitted into the patient, the returning acoustic signal travels through the spacer 92 to the acoustic detector 94.

In some embodiments, by providing the angled laser diode assembly 96 the likelihood that light will escape the spacer 92 when the light delivery device is removed from the surface of the patient's skin (i.e., surface 110 is no longer in contact with the surface of the patient's skin) may be significantly reduced or eliminated. This feature may offer advantages by decreasing the intensity of the light that escapes the sensor assembly 90 and/or by decreasing the likelihood that emitted light reaches the patient or others in the surrounding environment when the assembly is not positioned for use on tissue (e.g., when the sensor assembly is carried or lifted for repositioning by an operator).

More specifically, an angle 112 between the emitted light and a side surface 114 of the spacer 92 may be selected such that a light delivery angle is larger than the critical angle (i.e., the angle of incidence above which total internal reflection occurs) for the spacer 92 to air interface. The critical angle depends on the refractive indices (n) of the materials, and may generally be determined by the following equation:

θ₁=arcsin(n₂/n₁)

For example, in embodiments in which Rexolite is used as the spacer 92, the angle 112 may be selected such that the light delivery angle is larger than approximately 39 degrees, which is the critical angle for the Rexolite (n₁=1.57) to air (n₂=1.0) interface. In embodiments in which the angle 112 is in this manner, the emitted light will be totally internally reflected when the sensor assembly 90 is removed from the surface of the patient's tissue. Therefore, the emitted light will remain reflected within the sensor assembly, and will not emit into the surrounding environment, thereby reducing or eliminating the likelihood that an operator or others in the surrounding environment are exposed to the emitted light when the sensor assembly is removed from the patient. When the sensor assembly 90 is in contact with the patient's skin, the light delivery angle 112 is less than the critical angle due to the change of index of refraction at the spacer 92 to tissue (n₃=1.4) interface, and therefore the light is emitted into the patient tissue as desired for the PA response. An additional consideration is the critical angle between the spacer 92 and the patient's skin, and any angle greater than the critical angle will not pass through to the patient's skin. The angle 112 at which the light is emitted from the angled laser diode assembly 96 is preferably controlled between a first critical angle (e.g., the critical angle for the spacer to air interface) and a second critical angle (e.g., the critical angle for the spacer to tissue interface). In certain embodiments, the angle 112 is preferably controlled to approach or to be near the second critical angle so that a higher percentage of light enters the patient's tissue and a lower percentage of light is internally reflected when the spacer 92 is positioned on the patient's tissue. By way of example, if the spacer 92 is formed from Rexolite, the critical angle for the Rexolite to tissue interface is approximately 63 degrees. In some embodiments, the angle 112 of incidence is preferably controlled between approximately 39 and 63 degrees. In certain embodiments, the angle 112 is preferably controlled to approach 63 degrees (e.g., be approximately 63 degrees, between about 60 and 63 degrees, between about 55 and 63 degrees, between about 50 and 63 degrees, etc.) so that a higher percentage of light enters the patient's tissue and a lower percentage of light is reflected when the Rexolite spacer is positioned on the patient's tissue. Thus, the spacer 92 and the angled laser diode assembly 96 can be configured to facilitate total internal reflection of the emitted light when the sensor assembly 90 is not applied to the patient, but to enable transmission of the emitted light to the patient tissue when the sensor assembly 90 is applied to the patient's skin. Such a configuration may prevent emission of high intensity light unless the surface 110 is in contact with the patient's skin. Additionally or alternatively, the spacer 92 may be configured to allow the laser light to expand as it propagates through the spacer lowering the light density. In operation, after the light is transmitted to the patient's tissue, the PA signal may be received on the bottom surface 110 of spacer 92 and transmitted through the spacer 92 to acoustic detector 94.

The angled laser diode assembly 96 may be connected by a cable 116 to a power source and/or medical device. In other implementations, any of the disclosed embodiments provided herein may also be configured as wireless sensors. In certain embodiments, the sensor assembly 90 may include the housing 98 that fully or partially surrounds the spacer 92, the angled laser diode assembly 96, and/or the acoustic detector 94, for example. The housing 98 may be generally configured to protect the sensor assembly 90, and the housing 98 may take any suitable form and may be formed from any suitable materials. For example, in some embodiments, the housing 98 may be formed from plastic or metal materials, or any combination thereof. In some embodiments, the housing 98 may be opaque or may have a colored interior surface (e.g., dyed, painted, pigmented, etc.) configured to absorb light that escapes the spacer 92. The housing 98 may thus reduce the amount of light that escapes the sensor assembly 90. In some embodiments, an air gap 118 is provided between the spacer 92 and the housing 98 to reduce or prevent any acoustic signals from being generated at the housing surface where the light is absorbed. Additionally, the air gap 118 may provide a resistance so that any acoustic signal that is generated at the housing surface is not able to transmit through the air gap to the acoustic detector 94.

As set forth above, FIG. 2 depicts an embodiment of the sensor assembly 90 that is configured to control the emission of light by providing the angled laser diode assembly 96 to facilitate total internal reflection of the emitted light when the sensor assembly 90 is not applied to the patient's skin. Additionally or alternatively, the PA spectroscopy system 8 may include other light-controlling features. For example, the sensor assembly 90 may include or may be coupled to one or more sensing elements 26 that generate signals indicative of whether the sensor assembly 90 is positioned on the patient's skin, as discussed in more detail below. The system 8 may, in turn, be configured to operate and/or control the light source 18 based at least in part on whether the sensor assembly is determined to be on the patient's skin.

The sensing elements 26 may be used with any suitable sensor assembly, such as the sensor assembly 90, having the angled laser diode 96. Additionally, as shown in FIG. 3, the sensing elements 26 may be used in a sensor assembly 120 in which a laser diode assembly 122 is positioned so that the emitted light is approximately orthogonal (e.g., perpendicular, 90 degrees, etc.) to the patient's tissue. In some instances, during operation, this orthogonality may result in a reduced quantity of background noise as compared to non-orthogonal designs, as more light is absorbed into the skin and less is reflected, and the higher light density associated with such configurations may result in a stronger photoacoustic signal Additionally, the orthogonally emitted light has a higher light power density, resulting in a stronger acoustic signal. Additionally, the embodiment of FIG. 3 may offer certain advantages, such as enabling more control over a spot size of the emitted light that reaches the patient and accommodating a variety of sizes of acoustic detectors 124. The emitted light and acoustic waves may pass through a spacer 126.

With reference to FIG. 3, the sensing elements 26 may be coupled to the sensor assembly 120 in any suitable manner and may be disposed in any suitable location, such as on a bottom surface 128 of the sensor assembly 120. Although the sensor assembly 120 is shown without a housing, it should be understood that a housing, such as housing 98 in FIG. 2, may surround the spacer 126 and that an air gap, such as air gap 118, may be provided between the spacer 126 and the housing. In some embodiments, the sensing elements 26 may be recessed within the spacer 126. Such a configuration may protect the sensing elements 26 during use and/or may allow the sensing elements 26 to be flush (e.g., level) with the bottom surface 128 of the spacer 126, which in turn may aid in placing the sensor assembly 120 properly on the patient's tissue. Such a configuration may also enable the sensing elements 26 to accurately detect when the sensor assembly 120 peels away (e.g., lifts off, separates from, etc.) the patient's skin. In some embodiments, the sensing elements 26 may be coupled to a flex circuit or a plate (e.g., a metal plate) that is configured to be coupled to (e.g., removably attached or fixedly attached) to the sensor assembly 120 (e.g., the bottom surface 128 of the sensor assembly 120. In certain embodiments, the sensing elements 26 may be placed outside of a pathway 130, through which the emitted light and/or acoustic waves travel through the spacer 126. The sensing elements 26 may be positioned outside of the pathway 130 or in any position in which the sensing elements 26 do not interfere with the emitted light and/or the acoustic waves. Furthermore, any suitable sensing elements 26 may be utilized. For example, any sensing elements 26 configured to generate signals indicative of whether the sensor assembly 120 is off, on, and/or near the patient's skin may be used in the presently disclosed system 8. Thus, as set forth below, the sensing elements 26 may include impedance sensors, temperature sensors, pressure sensors, and the like. In some embodiments, the sensing elements 26 may comprise an optical detector configured to generate an optical signal that may indicate whether the sensor assembly 120 is positioned on the patient's tissue. In certain embodiments, the sensing elements 26 may broadly comprise the acoustic detector 124 detector configured to generate the acoustic signal that may indicate whether the sensor assembly 120 is positioned on the patient's tissue (e.g., that may be processed by the monitor 12 to determine whether sensor assembly 120 is positioned on the patient's tissue).

In some embodiments, the sensing elements 26 may include one or more electrical contacts or impedance sensors. In certain embodiments having such impedance sensors, two or more electrodes may be positioned on a bottom surface of the sensor assembly 120. The system 8 may be configured to measure a voltage corresponding to a current flowing between the electrodes, the measured voltage being proportional to the impedance between the electrodes. Since the impedance of the air surrounding the sensor assembly 120 is higher than the impedance of the surface of the patient's skin, the measured voltage will be relatively low when the electrodes are placed firmly against the patient's skin, and the voltage will be relatively high when both electrodes are exposed to the air (e.g., when the electrical path between the electrodes is through the air). Thus, the measured voltage may be compared to a certain threshold voltage to determine whether the electrodes of the sensing elements 26 (and thus the sensor assembly 120) are applied to the patient's skin. Any of a variety of suitable types of sensing elements 26, such as thermistors or temperature sensors, may be utilized to detect whether the sensor assembly 120 is positioned on the patients skin via various sensed parameters (e.g., temperature). Additionally, various combinations of one or more different types of sensing elements 26 may be used with the sensor assembly 120. Thus, in some embodiments, two or more different types of sensing elements 26 may be provided and/or coupled to the sensor assembly 120 to provide signals indicative of whether the sensor assembly 120 is positioned one the patient's skin.

The sensing elements 26 may be spaced or located on the PA sensor assembly 120 in accordance with the intended orientation of the PA sensor on the patient. PA sensor assemblies, such as the sensor assembly 120, may be placed in any suitable location on the patient 24. As shown in FIG. 4, in certain embodiments, the sensor assembly 120 may be placed on or near the patient's temple. In some such embodiments, the sensor assembly 120 may include a support structure 140 (e.g., clip, hook, arm, etc.) configured to couple the sensor assembly 120 to the patient's ear. The sensor assembly 120 may be relatively heavy compared to other types of medical sensors (e.g., pulse oximeters), and thus the sensor assembly 120 may have a tendency to separate from the patient's skin. In some configurations, the sensor assembly 120 may have a tendency to separate from the patient's skin at the corners or edges of the sensor assembly 120. For example, when the patient is standing or in an upright position, a top edge 142 (e.g., first edge) may have a tendency to separate from the patient's skin, and thus, it may be desirable to provide sensing elements 26a, 26b near or adjacent to the top edge 142 so that separation of the sensor assembly 120 from the patient's skin is detected as early as possible during should the sensor assembly 120 separate or dislodge from the patient's skin. In certain embodiments, the sensor assembly 120 may be configured to be applied to a supine patient, and thus, sensing elements 26b, 26c may desirably be positioned near or adjacent to a front edge 144 (e.g., second edge), as the sensor may be more likely to separate at the front edge 144 first when the patient is lying down. In some embodiments, the sensing elements 26a, 26b, 26c, 26d may be positioned at or near each corner of a generally rectangular bottom surface 128 of the sensor assembly 120. The sensing elements 26 may also have any shape or form, and thus, the sensing elements 26 may be arranged or formed into strips or lines that are positioned adjacent to one or more edges of the sensor assembly 120, for example.

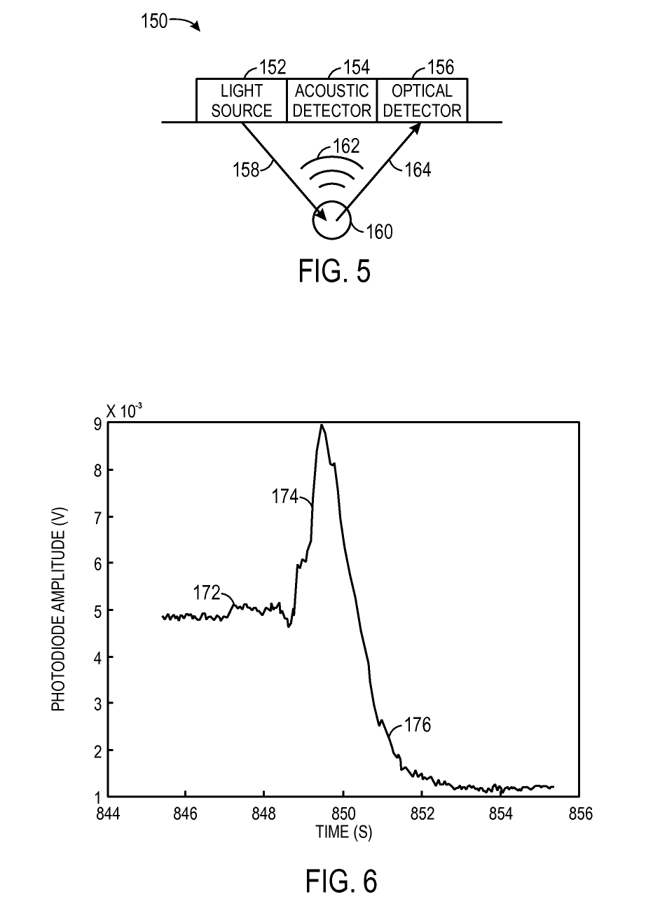

Additionally or alternatively, the system 8 may be configured to determine whether the sensor assembly 120 is positioned on the patient's skin through other techniques, such as an optical technique. As shown in FIG. 5, a sensor assembly 150 may include an optical detector 156 as well as a light source 152 and an acoustic detector 154. When the sensor assembly 150 is applied to a patient, the light source 152 directs light 158 toward a target blood vessel 160, in a manner as described above. A portion of the light 158 is absorbed in the blood vessel 160 to generate acoustic waves 162. Another portion 164 scatters toward the optical detector 156 and is detected. The acoustic detector 154 and the optical detector 156 generate signals representative of the detected acoustic waves 162 and the detected light 164, respectively.

In accordance with certain embodiments, the signals generated by the optical detector 156 may be utilized by the system 8 to determine whether the sensor assembly 150 is positioned on the patient's skin. Any suitable processing method for determining signal quality may be employed to assess the quality of the received signal and to determine if the signal meets a certain threshold quality. For example, the signal may be qualified based on a pulse qualification. Through such techniques, the signal may be evaluated to identify a pulse of blood due to a heart beat, and the presence of such a pulse may indicate that the sensor assembly 150 is on the skin. In some embodiments, the signal may be qualified based on a ratio of ratios of the signal. Through such techniques, the signal may be evaluated to determine an oxygen saturation measurement, which may indicate that the sensor is properly placed over an artery on the skin and may be used by the system 8 to determine that the sensor assembly 150 is positioned on the patient's skin. Signal quality assessments and signal qualification may be performed via any suitable technique, such as the techniques provided in U.S. Pat. No. 7,209,774, the specification of which is incorporated by reference herein in its entirety herein for all purposes.

As set forth above, the system 8 may be configured to determine whether the sensor assembly 150 is applied to the patient's skin based at least in part on the signal qualification. For example, the system 8 may determine that the sensor assembly 150 is applied to the patient's skin if various metrics related to the received signal or characteristics of the received signal meet certain minimum thresholds or is otherwise determined to be qualified. In some embodiments, the system 8 may determine and/or qualify or evaluate various metrics that quantify one or more aspects of the signal. The system 8 may determine and/or evaluate various metrics such as a heart rate, an average pulse period, an amplitude of the signal, a ratio of ratios, a pulse shape (e.g., skew of the signal or skew of the derivative), and/or a frequency of the signal. For example, the system 8 may compare a calculated heart rate metric to certain thresholds, such as a minimum heart rate of 35, 40, 50, or more beats per minute (bpm). Thus, by way of example, if system 8 calculates a heart rate lower than 50 bpm, then the system 8 may determine that the sensor assembly 150 is not applied to the patient's tissue. The threshold may be set at a manufacturing stage, may be adaptively set prior to or during a monitoring session for each patient based on historical data, and/or may be set or adjusted by a user based on user preferences and/or patient characteristics. In certain embodiments, the system 8 may determine that the sensor assembly 150 has become dislodged from the skin and/or is not positioned on the patient's skin if the metric (e.g., the heart rate metric, the amplitude, etc.) changes by more than 1%, 5%, 10%, 15%, 20% or more over a period of time (e.g., 1, 2, 3, 4, 5, or more seconds) during a monitoring session. Again, such tolerances or metrics may be set at a manufacturing stage, may be adaptively set prior to or during a monitoring session for each patient based on historical data, and/or may be set or adjusted by a user based on user preferences and/or patient characteristics, for example. The determination of whether the signal is qualified may trigger certain actions, as described in more detail below.

The above-described technique may be used with light sources 152 that emit a single wavelength of light (e.g., light in a single spectrum, such as light in the IR spectrum) or that emit two or more wavelengths of light (e.g., light in the IR and the red spectrum). If the light source 152 is configured to emit two wavelengths of light, the system 8 may additionally be configured to generate plethysmography (PPG) signals and to determine various types of physiological information (e.g., oxygen saturation, etc.). The PPG signal may also be qualified via the techniques described above to determine whether the sensor assembly 150 is applied to the patient's skin.

However, as shown in FIG. 4, in certain embodiments, the sensor assembly 120 may be placed on a temple of the patient or over a large artery. Such locations are typically not well-suited for pulse oximetry or for determining oxygen saturation based on the PPG signal. Furthermore, certain light sources 152 may be bulky and or heavy. Thus, in some embodiments, it may be desirable for the light source 152 to emit only one wavelength of light (e.g., light in a single spectrum, such as the IR spectrum), and the system 8 may be configured to qualify the signal generated by the optical detector using only one wavelength of light via the techniques described above and/or the techniques described below. Such a configuration may use less hardware and/or may reduce processing steps, while enabling qualification of the optical signal for determining whether the sensor assembly 150 is applied to the patient's skin.

In some embodiments, the optical detector 156 may additionally or alternatively be configured to monitor an optical reflectance of light from the patient's skin. In particular, the optical detector 156 may receive the light reflected from the patient's tissue, and the system 8 may be configured to monitor a direct current (DC) component of the optical signal to determine whether the sensor assembly is positioned near the patient's skin. FIG. 6 is a plot of an example of a DC component 170 of the optical signal as the sensor (e.g., photoacoustic sensor assembly 150 having an optical detector 156) transitions from a position on a patient's skin to a position off of the patient's skin, in accordance with an embodiment. The DC component 170 of the optical signal is a non-pulsatile component of the signal and is the result of light absorption by nonpulsatile tissue, such as fat, bone, muscle, and skin. As shown, the sensor assembly 150 is initially on the patient's skin at a first time 172. The DC component 170 is expected to increase sharply as shown at a second time 174 when the sensor assembly 150 initially peels away (e.g., lifts off, separates from, etc.) and then to decrease as shown at a third time 176 as the sensor assembly 150 moves away from the patient's skin. The changes in the DC component 170 of the optical signal are due, at least in part, to the increased in light reflected from the surface of the skin to the optical detector 156 when the sensor assembly 150 is a first distance (e.g., a short distance) away from the skin, as the sensor assembly 150 moves to a second distance (e.g., a far distance, greater than the first distance) less emitted light reflects off the skin and/or less light reflected from the skin reaches the optical detector 156. Thus, the monitor 12 may be configured to monitor absolute values of the DC component 170 and/or to monitor relative changes of the DC component 170 over time to determine whether the sensor assembly 150 is applied to the patient's tissue. If the DC component 170 exceeds a certain threshold and/or if a certain or expected pattern (e.g., a sharp increase in the signal followed by a decrease in the signal) in the DC component 170 is detected, the system 8 may determine that the sensor assembly is not positioned on the patient's skin. In some embodiments, if the amplitude of the DC component decreases by more than about 1, 2, 3, 4, 5, 6, 7, 8, 9, 10, or more percent within 1, 2, 3, 4, 5, 6, 7, 8, 9, 10, or more seconds, the system 8 may determine that the sensor assembly 150 is not positioned on the patient's skin. In certain embodiments, if the amplitude of the DC component changes by more than about 5 or more than about 10 times its value within about 1, 2, or 3 seconds, the system 8 may be configured to determine that the sensor assembly 150 is not positioned on the patient's skin. Again, such tolerances or thresholds may be set at a manufacturing stage, may be adaptively set prior to or during a monitoring session for each patient based on historical data, and/or may be set or adjusted by a user based user preferences and/or patient characteristics, for example.

While the illustrated embodiment of FIG. 5 shows a sensor arrangement in which the acoustic detector 154 is between the light source 152 and the optical detector 156, it should be understood that other arrangements are contemplated. For example, the optical detector 156 may be positioned adjacent to the acoustic detector 154 but not along an axis connecting the light source 152 and the acoustic detector 154. In certain embodiments, the optical detector 156 and the acoustic detector 154 are directly next to or adjacent to one another. In one example of such an arrangement, the housings or support structures for these elements may contact one another. In this manner, the negative correlation between their signals may be enhanced. In another embodiment, the optical detector 156 and the acoustic detector 154 are spaced apart from one another. Additionally, while only one light source 152 is illustrated in FIG. 5, it should be understood than any suitable number of light sources 152 may be used in the sensor assembly 150. The geometry of the arrangement of the optical and acoustic components on the sensor assembly 150 may influence the calibration of the sensor and may be provided as an input to certain algorithms. Accordingly, in one embodiment, sensor geometry information as well as other sensor identification information and/or calibration information may be stored in the encoder 28.

Additionally or alternatively, the system 8 may be configured to determine whether the sensor assembly 150 (or sensor assembly 120, for example) is positioned on the patient's skin through other techniques, such as acoustic techniques. For example, in certain embodiments, sensor assembly 150 may be configured to monitor an intensity of the acoustic signal 162 and/or changes in the acoustic signal 162 and to determine whether the acoustic signal 162 indicates that the sensor assembly 150 is not on the patient's skin. In certain embodiments, the system 8 may be configured to determine whether sensor 10 is applied to the patient's tissue based on the intensity of the acoustic signal 162 or changes in the acoustic signal 162 over a period of time. The system 8 may be configured to monitor a quality of the acoustic signal 162 based on various parameters or features of the received signal (e.g., amplitude, shape, etc.). For example, sudden diminishment (e.g., decrease in amplitude) of the acoustic signal 162 may indicate that the sensor assembly 150 has become dislodged and/or is not positioned on the patient's skin. In certain embodiments, if the acoustic signal 162 decreases by more than about 1, 2, 3, 4, 5, 6, 7, 8, 9, 10, or more percent within 1, 2, 3, 4, 5, 6, 7, 8, 9, 10, or more seconds, the system 8 may determine that the sensor assembly 150 is not positioned on the patient's skin or has dislodged (e.g., peeled off) the patient's skin. As noted above, such tolerances or thresholds may be set at a manufacturing stage, may be adaptively set prior to or during a monitoring session for each patient based on received signals or patient data, and/or may be set or adjusted by a user based on user preferences and/or patient characteristics, for example.

The above-described embodiments relate generally to photoacoustic sensor systems 8 that are configured to determine whether the sensor 10 (or sensor assembly 90, 120, 150, for example) is applied to the patient's skin. The system 8 may also be configured to trigger an appropriate response based at least in part on whether it is determined that the sensor 10 is applied to the patient's skin. For example, the system 8 may determine whether the sensor 10 is applied to the skin using one or more of the above techniques, and the information may be provided to a controller that is configured to control the operation of the light source 18.

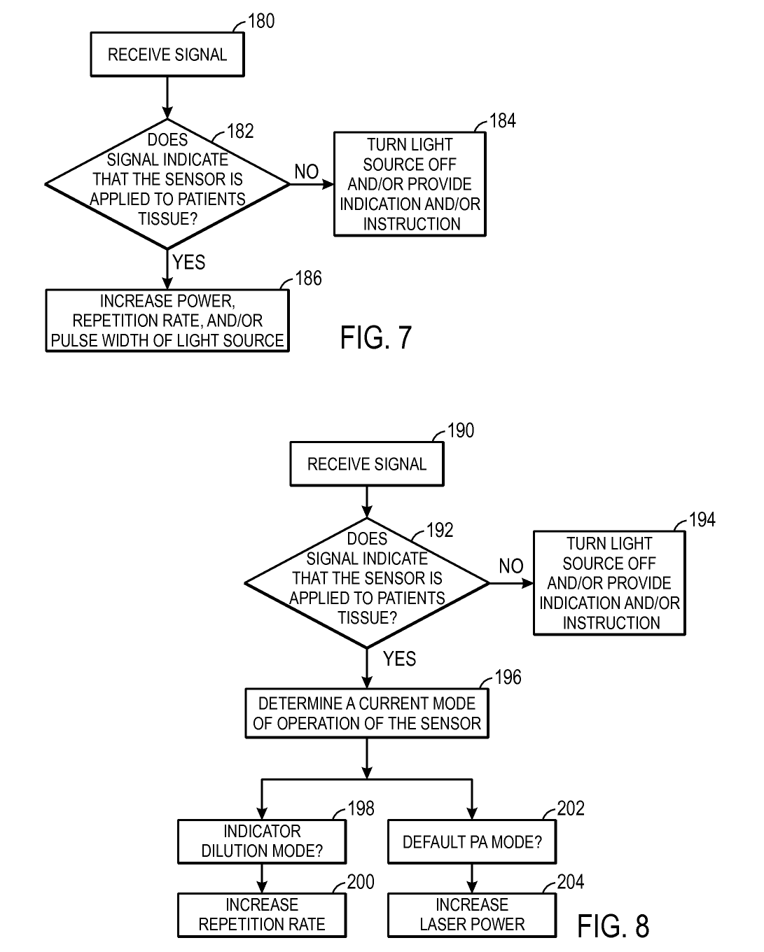

FIG. 7 is a flow diagram of one technique for operating the sensor 10 and/or controlling the light source 18 in accordance with the present disclosure. As shown, at step 180, the signal indicative of whether the sensor 10 is positioned on the patient's tissue is received (e.g., at the microprocessor 32). As indicated above, the signal may be a voltage or current signal generated by the sensing elements 26, an optical signal generated by the optical detector 22, or an acoustic signal generated by the acoustic detector 20, for example. At step 182, the system 8 processes the signal to determine whether the sensor 10 is positioned on the patient's skin, based on the techniques described above for each type of signal. As shown in steps 184 and 186, certain actions are triggered based on whether it is determined that the sensor 10 is positioned on the patient's skin. For example, if it is determined that the sensor is not on the patient's skin, the system 8 may disable the light source 18 as shown in step 184. In certain embodiments, the system 8 may additionally or alternatively decrease an intensity, a repetition rate, and/or a pulse width of the light emitted by the light source 18 if it is determined that the sensor is not on the patient's skin or has become separated from the patient's skin. In some embodiments, the system 8 may provide an indication that the sensor 10 is not on the patient's skin (e.g., an audible or visual alarm, a message on the display 14) and/or may provide instructions to the user to correct the issue, such as instructions to adjust the sensor, to firmly press the sensor to the patient's skin, to replace a disposable portion of the sensor 10, to replace all of the sensor 10, or to clean the patient contact surfaces, for example.

In certain embodiments, if it is determined that the sensor 10 is on the patient's skin, then the system 8 may turn on the light source 18 as shown in step 186. In some embodiments, the light source 18 may initially run at low power or at a low repetition rate (e.g., about 50 Hz, 100 Hz, etc.) when the sensor 10 is turned on and/or prepared for placement on the patient, and the system 8 may increase the intensity, the repetition rate, and/or the pulse width of the light emitted by the light source 18 when it is determined that the sensor 10 is applied to the patient's skin. In some embodiments, the system 8 may provide an indication or confirmation that the sensor 10 is applied to the patient's skin (e.g., an audible or visual alarm, a message on the display 14).

FIG. 8 is a flow diagram of another technique for operating the sensor 10 and/or for controlling the light source 18 in accordance with the present disclosure. In certain embodiments, the appropriate response may depend on a type of PA spectroscopy system 8 and/or a current mode of operation of the system 8. Accordingly, in some embodiments, the system 8 may additionally determine the type of system 8 (e.g., a system configured to monitor oxygen saturation, a system configured to monitor indicator dilution, etc.), and/or the current mode of operation of the system 8 (e.g., the current mode of operation of a system having multiple modes, such as a default mode, an indicator dilution mode, etc.). Such a determination may be made by accessing information (e.g., operating parameters or capabilities, sensor identification data, such as sensor model number, etc.) stored in the sensor 10, such as in the encoder 28. In some embodiments, the current monde of operation may be input by a user. As shown in FIG. 8, at step 190, a signal indicative of whether the sensor 10 is positioned on the patient's tissue is received (e.g., at the microprocessor 32). The signal may be generated by the sensing elements 26, by the optical detector 22, or by the acoustic detector 20, for example. At step 192, the system 8 processes the signal to determine whether the sensor 10 is positioned on the patient's skin. As shown in step 194, if the sensor 10 is not applied to the patient's skin, the system 8 may turn the light source 18 off or may adjust the laser power, pulse rate, and/or pulse width so that the light is emitted at an eye-safe level. Such adjustments may be carried out by controlling the drive current of the laser or by controlling the AOM 25, for example. The system 8 may additionally or alternatively take any another suitable action, such as providing an indication that the sensor 10 is not on the patient's skin. As discussed above, the system 8 may also provide instructions, such as instructions to adjust the sensor, to firmly press the sensor to the patient's skin, to replace a disposable portion of the sensor 10, to replace the whole sensor 10, or to clean the patient contact surfaces, for example.

If the signal indicates that the sensor 10 is applied to the patient's tissue, the system may proceed to step 196. At step 196, the system 8 may determine a current mode of operation of the sensor 10. For example, the system 8 may determine whether the system 8 is operating in a default PA mode, an indicator dilution mode, or any other suitable mode. If the sensor 10 is determined to be on the patient's skin and the system 8 is operating in an indicator dilution mode as shown in step 198, the system 8 may increase the repetition rate of the light emitted by the light source 18 as shown in step 200. For example, the sensor 10 may initially run at a very low repetition rate in preparation for patient monitoring, and the system 8 may increase the repetition rate (e.g., from about 50 Hz-100 Hz to about 1 kHz) if it is determined that the sensor 10 is on the patient's skin. However, if the sensor 10 is determined to be on the patient's skin and the system 8 is operating in a default PA mode as shown in step 202, the system 8 may increase the power of the light emitted by the light source 18 as shown in step 204. For example, the drive current may be increased to increase the power of the light.

It should be noted any of the methods provided herein, may be performed as an automated procedure by a system, such as system 8. Although the flow charts illustrate the steps in a certain sequence, it should be understood that the steps may be performed in any suitable order and certain steps may be carried out simultaneously, where appropriate. For example, with reference to FIG. 8, the system 8 may first determine a current mode of operation of the sensor 10, and then proceed to determine whether the signal indicates that the sensor is applied to the patient's tissue. Further, certain steps or portions of the methods may be performed by separate devices. In addition, insofar as steps of the methods disclosed herein are applied to the received signals, it should be understood that the received signals may be raw signals or processed signals. That is, the methods may be applied to an output of the received signals.

Certain described embodiments relate generally to features that generate signals indicative of whether the sensor 10 is applied to the patient's skin. It should be understood that in other embodiments, the sensor 10 may additionally or alternatively include other features, such as a switch. The switch may be a mechanical switch and may be positioned at any suitable location on the sensor 10. For example, the switch may be positioned on a bottom surface of a spacer, such as the bottom surface 128 of the spacer 126 of FIG. 3. The switch may prevent the light source 18 from emitting light unless the sensor 10 is applied to the patient's skin. More particularly, the switch may be closed when the sensor 10 is applied to the patient's skin and open when the sensor 10 is not applied to the patient's skin, or vice versa. Thus, for example, the switch may mechanically disable the light source 21 when the sensor 10 is not applied to the patient's skin, and thus may be a light-controlling feature.

The disclosed embodiments are provided in the context of a photoacoustic spectroscopy system. However, it should be understood that the features described herein may be incorporated into any sensor assembly having a high-intensity light source. Furthermore, the various features and techniques described herein may be combined or utilized together in any suitable manner to determine whether a medical sensor, such as a photoacoustic sensor, is applied to the patient's skin and to appropriately control the light source. While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A photoacoustic system, comprising:
a photoacoustic sensor assembly, comprising:
a light emitting component configured to emit one or more wavelengths of light into a region of a patient's tissue; and
an acoustic detector configured to detect acoustic energy generated within the region of the patient's tissue in response to the emitted light, wherein the sensor assembly is configured to generate a signal that enables a monitor to determine a position of the sensor assembly relative to the patient's tissue.

2. The photoacoustic system of claim 1, comprising an electrical contact sensor configured to generate the signal.

3. The photoacoustic system of claim 2, wherein the electrical contact sensor is positioned outside of a pathway through which the acoustic energy is transmitted from the patient's tissue to the acoustic detector.

4. The photoacoustic system of claim 1, comprising an optical detector configured to receive light emitted by the light emitting element and to generate the signal based on the received light.

5. The photoacoustic system of claim 1, wherein the light emitting component is oriented such that emitted light contacts the patient's tissue at a non-orthogonal angle with respect to the patient's tissue when the sensor is applied to the patient's tissue.

6. The photoacoustic system of claim 1, comprising the monitor coupled to the photoacoustic sensor assembly, wherein the monitor comprises a memory storing instructions for:
receiving the signal from the photoacoustic sensor assembly;
determining the position of the photoacoustic sensor assembly based at least in part on the signal received from the photoacoustic sensor assembly; and
adjusting a light drive signal to the light emitting component based at least in part on the position.

7. The photoacoustic system of claim 6, wherein the adjusting comprises reducing an intensity of the light emitted by the light emitting component.

8. The photoacoustic system of claim 6, further comprising providing an indication of the position.

9. The photoacoustic system of claim 1, wherein the signal comprises the detected acoustic energy.

10. A photoacoustic monitoring system, comprising:
a photoacoustic sensor assembly configured to emit light;
a monitor comprising:
a memory storing instructions for:
receiving a signal from the sensor assembly indicative of a proximity of the sensor assembly to a tissue of a patient;
determining whether the sensor assembly is positioned on the tissue of the patient based on the signal; and
adjusting the emitted light based at least in part on the determination; and
a processor configured to execute the instructions.

11. The photoacoustic monitoring system of claim 10, wherein the sensor assembly comprises an optical detector configured to receive the emitted light and to generate the signal indicative of the proximity of the sensor assembly to the tissue of the patient, wherein the memory stores instructions for qualifying the signal to determine whether the sensor assembly is applied to the tissue of the patient.

12. The photoacoustic monitoring system of claim 10, wherein the photoacoustic sensor assembly comprises an acoustic detector configured to detect the emitted light after the emitted light passes through the patient's tissue and to generate an acoustic signal in response to the detected light, wherein the signal indicative of the proximity of the sensor assembly to the tissue of the patient comprises the acoustic signal.

13. The photoacoustic monitoring system of claim 10, wherein the memory stores instructions for increasing the power if it is determined that the sensor assembly is applied to the tissue of the patient.

14. The photoacoustic monitoring system of claim 10, wherein the memory stores instructions for increasing the pulse width of the emitted light if it is determined that the sensor assembly is applied to the tissue of the patient.

15. The photoacoustic monitoring system of claim 10, wherein the memory stores instructions for disabling the light emitting component if it is determined that the sensor assembly is not applied to the tissue of the patient.

16. The photoacoustic monitoring system of claim 10, wherein the memory stores instructions for determining whether the sensor assembly is operating in an indicator dilution mode.

17. The photoacoustic monitoring system of claim 16, wherein the memory stores instructions for increasing the repetition rate of the emitted light if it is determined that the sensor assembly is operating in the indicator dilution mode and that the sensor assembly is applied to the tissue of the patient.

18. A method, comprising:
emitting one or more wavelengths of light from a light source of a sensor assembly into an interrogation region of a patient;
detecting an acoustic response to the emitted light from the interrogation region of the patient with an acoustic detector of the sensor assembly;
processing a signal indicative of whether the sensor assembly is positioned on a tissue of the patient; and
determining whether the sensor assembly is positioned on the tissue of the patient based on the processed signal.

19. The method of claim 18, wherein the signal indicative of whether the sensor assembly is positioned on the tissue comprises the detected acoustic response.

20. The method of claim 18, comprising determining a current mode of operation of the sensor assembly and triggering a response based at least in part on the current mode of operation and based at least in part on whether the sensor assembly is positioned on the tissue of the patient.

21. The method of claim 20, comprising increasing the repetition rate if it is determined that the current mode of operation is an indicator dilution mode and the sensor assembly is positioned on the tissue of the patient.

22. The method of claim 20, comprising increasing the power of the light if it is determined that the current mode of operation is a default photoacoustic mode and the sensor assembly is positioned on the tissue of the patient.
